(19) United States
(12) Patent Application Publication
(10) Pub. No.: US 2005/0136134 A1
(43) Pub. Date: Jun. 23, 2005

(54) Composition for the control of pathogenic microorganisms and spores

(75) Inventors: Murray Royal Bast, Wellesley (CA); Raul Cuero, Cypress, TX (US); Dana Lee Eccleston-McGregor, Tavistock (CA)

Correspondence Address: PATTON BOGGS LLP, 8484 WESTPARK DRIVE, SUITE 900, MCLEAN, VA, 22102, US

(73) Assignee: Bio-Ag Consultants & Distributors, Inc., Wellesley (CA)

(21) Appl. No.: 10/740,514

(22) Filed: Dec. 22, 2003

(51) Int. Cl. A61K 35/78; A61K 31/555; A61K 31/28

(52) U.S. Cl. 424/736; 424/750; 424/766; 514/492; 514/184

(57) ABSTRACT

A safe, environmentally-friendly anti-microbial composition is provided. The composition comprises an extract of barley and chelated mineral ions. A grape or lemon extract is optionally included. The composition is useful as a surface decontaminant or as a feed component.

### FIELD OF THE INVENTION

The present invention relates to the control of pathogenic microorganisms. In particular, a safe, environmentally friendly antimicrobial composition is provided.

### BACKGROUND OF THE INVENTION

There is a longstanding need for antimicrobial agents having improved antimicrobial activity and improved speed of action. In addition to the desire to control naturally occurring infectious diseases, there is mounting concern about biological warfare.

The specific requirements for antimicrobial agents vary according to the intended use. For example, antimicrobial agents are used in sanitizers, disinfectants, sterilizers, aseptic packaging, etc. There is a large demand for antimicrobials that are safe for use in the food industry. In addition, antimicrobial compositions find many uses in the field of human health where it is known that the severity of disease is related to the number of pathogenic organisms to which a person is exposed.

Various agents, such as alcohol, phenols, hypochlorites, chlorine, ammonium, ozone, etc., have been described which have a broad spectrum of antimicrobial properties. However, many of these agents have significant drawbacks. Some agents have inadequate activity and others are toxic or are not environmentally friendly. In addition, many of the current antimicrobial compositions require significant time to take effect and some microorganisms can develop resistance to these antimicrobial compounds, especially after a long period of time. Furthermore, many of these antimicrobial compounds are not cost effective.

Certain metal salts have been shown to have antibacterial activity. For example, U.S. Pat. No. 2,946,722 discloses that a combination of sodium propionate, methyl rosaniline, ferric choline citrate, menadione and trace elements provide a varied coverage of antibacterial, antifungal, anti-hemorrhagic and stress compensating factors in a well balanced formula. U.S. Pat. No. 4,938,955 discloses an antibiotic resin that comprises antibiotic metal ions. U.S. Pat. No. 6,139,879 discloses heavy metal chelate compositions that are useful as fungicides and bactericides.

There remains, however, a need for an antimicrobial composition that is active against both gram positive and gram-negative organisms including sporulating bacteria. Bacillus anthracis is one example of sporulating bacteria. The need for an agent that is effective against spores is particularly pressing in view of the recent transmission of anthrax spores through the mail. Humans become infected with Anthrax by coming into contact with the spores through the respiratory tract, skin, mucous membranes and the digestive tract. Spores can survive for over 50 years and biological warfare attack spores can be transmitted through the air, food, water or any kind of contact. Sporulating gram-positive bacteria are also frequent contaminants in food and lead to gastrointestinal problems. Gram-negative rods, such as certain members of the Enterobacteriaceae family, are among the most pathogenic and most encountered organisms in clinical microbiology. They are the causative agents of diseases such as meningitis, dysentery, typhoid and food poisoning. Thus, there remains a real need for a versatile antimicrobial agent that is safe for humans and which is active against a wide variety of microorganisms and spores.

### SUMMARY OF THE INVENTION

The present invention addresses the need for an antimicrobial composition that is environmentally safe and can be applied effectively to animals and humans. Food and beverages can be treated with the composition to prevent the spread of disease. The composition can be used as a preventative or to treat meats and other liquid or solid foods contaminated with bacteria, bacterial spores and/or fungi. The composition is effective against both gram positive and gram-negative organisms. The composition can also be used to decontaminate surfaces. The present invention provides a cost effective technology that is reliable and easy to use.

It is an object of one aspect of the present invention to provide an antimicrobial composition for the surface decontamination of food such as meat, fish, poultry, fruits and vegetables as well as beverages and water caused by various bacteria, yeast and fungi. The composition of the present invention is also useful for treating animals and humans to control and/or reduce the presence of pathogenic bacteria, yeast and fungi.

In one aspect of the invention, an antimicrobial composition is provided. The antimicrobial composition comprises an extract of barley. The composition optionally includes at least one chelated metal salt and/or a lemon extract.

In a preferred embodiment, the barley extract is derived from organically grown barley.

In another preferred environment, the chelated metal salt is a salt of a metal selected from the group consisting of iron, zinc, copper, cobalt, magnesium and manganese and mixtures thereof. Iron is a particularly preferred metal. By combining the chelated metal salts with the barley extract, the amount of chelated metal salts can be reduced. There is a synergistic effect whereby a concentration of chelated metal ions that would not normally be effective shows excellent antimicrobial activity. Low amounts of iron, as much as half of that normally used, have been found to be particularly effective, thus providing an economic advantage. For fungi, iron levels, as compared to other preparations, can be reduced by as much as three quarters.

In another preferred embodiment of the invention, the ratio of the chelated metal ions to the extract of the barley is selected from 1:1, 2:1, and 3:1. The concentrations of the components can be adjusted to prevent contamination or to control microbial growth. In a particularly preferred embodiment, the ratio of chelated mineral ions to extract of barley is 3:1.

In another preferred embodiment, the antimicrobial composition is provided as an antimicrobial concentrate. The working concentration of the composition can be adjusted depending on the intended use. Generally speaking, decontamination of solid substrates requires a higher concentration of antimicrobial. For solid surfaces, the composition of the present invention preferably contains 10% to 20% of the active ingredients. For treatment of liquids, a more dilute concentration may be used since it is more easily dispersed. A 5 to 10% concentration has been demonstrated to be effective in liquid cultures.

In another aspect of the invention, an antimicrobial composition is provided which comprises at least one chelated metal salt and an extract of barley in amounts that provide a greater than additive antimicrobial action in vitro.

In yet another aspect of the invention, a method for sanitizing a surface is provided. The method comprises applying an antimicrobial composition comprising at least chelated metal salt and a barley extract to a surface. The decontamination of various types of surfaces is contemplated. For example, the surface may be the surface of a food such as a meat, poultry, fish, fruits or vegetables. It may also be a solid surface such as a food handling table, sink, bath, toilet, floor and other surfaces. In addition, the surface may be the surface of an animal or human. This includes skin surfaces, tooth surfaces, and intestinal surfaces. The antimicrobial composition of the present invention can also be used to decontaminate liquids such as contaminated water and to prevent contamination of other liquids such as beverages. The antimicrobial composition can be applied as a liquid, spray, or coating. The composition may further include a carrier or diluents to enhance its application.

In another aspect of the invention, the antimicrobial composition may be administered as part of a foodstuff. A method of reducing the population of certain pathogenic bacteria in chicken eggs is also provided. This method comprises feeding the antimicrobial composition to laying hens. In addition, a method of reducing the population of pathogenic bacteria in animal manure is also provided. The amount of pathogenic bacteria in manure is reduced by feeding animals with the antimicrobial composition of the present invention.

In yet another aspect of the invention, a kit comprising an antimicrobial concentrate and instructions for diluting the concentrate is provided. Instructions for the use of the diluted antimicrobial composition are also provided. The composition and methods of the present invention offer many advantages over other known compositions and methods. In the present invention, the use of a mixture of non-toxic choline citrate chelated mineral ions in combination with a barley extract uses components that are easy to find commercially and in nature. In addition, the methods and compositions are environmentally safe. Furthermore, the technology can be applied effectively to animals and humans infected with pathogenic bacteria and/or fungi, as well as to meats and liquid or solid foods contaminated with bacteria and/or fungi. The technology can also be applied to contaminated solid surfaces such as tables, utensils, etc. It can also be applied to agriculture and horticulture. For example, the composition can be applied to prevent bacterial root rot. The present invention is useful in any situation where there is a desire to reduce microorganisms.

The present invention provides a rapid reduction in pathogenic bacteria within one hour and it is a cost effective technology. In addition, the technology is reliable in that the infection results are very consistent. The technology is also easy to apply and there is no need to use heat to speed up the process of removal of microorganisms.

### BRIEF DESCRIPTION OF THE DRAWINGS

FIG. 1 is a bar graph illustrating the bacterial levels in hogs treated with an antimicrobial composition according to the present invention;

FIG. 2A is a photograph illustrating a control bacterial culture under high power dark field microscopy;

FIG. 2B is a photograph illustrating a treated bacterial culture under high power dark field microscopy;

FIG. 3A is a photograph illustrating the effect of the anti-microbial composition in liquid culture of Bacillus subtilis;

FIG. 3B is a photograph illustrating a control Bacillus subtilis culture under high power dark field microscopy; and

FIG. 3C shows a treated Bacillus subtilis culture under high power dark field microscopy.

### DETAILED DESCRIPTION

The present invention provides antimicrobial compositions comprising an extract from barley. A complex of low concentrations of chelated mineral ions is optionally admixed with the barley extract. The compositions are safe and cost effective. The compositions provide a potent antimicrobial agent that can be used in animal and human health as well as in a variety of food handling operations. The compositions are also useful in a variety of domestic and industrial processes.

The present invention demonstrates that when barley extract is included in the formulation, animals and humans can be treated with an effective low concentration of chelated metal ions. As used herein, the term “chelate” refers to a coordination compound in which a central metal ion is attached by coordinate links to two or more non-metal atoms in the same molecule thus forming a heterocyclic ring. The molecule comprising the non-metal linking atoms is termed a chelator. One chelator useful in the preparation of the compositions of the present invention is choline citrate. It is clearly apparent that other metal chelators can be used to achieve the same effect.

The antimicrobial effectiveness of the chelated metal ions is augmented by the addition of an extract of barley to the composition. In a preferred embodiment, choline citrate chelated metal ions in combination with an extract of barley are used to control and/or reduce the presence of pathogenic bacteria and fungi. While a composition of metal ions alone is somewhat effective, the effect is enhanced when the barley extract is included. The combination of barley extract and ions provides consistent results. The combination is able to not only kill existing microorganisms and spores but also inhibit new growth.

The choline citrate chelated metal ions of the present invention are preferably based on iron, zinc, copper, cobalt, manganese, and magnesium. However, it is clearly apparent that other chelated metal salts (e.g. sulfate or chloride salts) could also be used. In a preferred embodiment a sulfate salt is used. A commercially available liquid, such as Hemoplex™, which supplies iron, copper, and cobalt synthesized in predetermined amounts to the choline citrate molecule, is a convenient source of chelated mineral ions for use in the present invention.

The barley extract of the present invention is preferably an extract derived from the leaves and/or roots of organically grown barley. The barley may be grown hydroponically. A screw or worm type process is typically used to extract the fluids that are then coarsely filtered. The present invention is not limited to the method used to extract the liquids, but encompasses other methods for extracting the fluids from barley that will be clearly apparent to those skilled in the art.

The antimicrobial compositions may also include lemon extract, grape extract, and/or essential oils. Commercial lemon juice may be used or an extract may be prepared by from the natural lemon fruit and optionally combined with sterile R/O water. Certain components from the skin (e.g. essential oils) may be included. When citric acid is included in the composition, it inhibits oxidation and helps to keep foods looking fresher. Ascorbic acid is also effective in the composition and a concentration of 0.5 to 2.0% ascorbic acid has been shown to be effective on fish and meat. Various combinations of barley extract and other components are contemplated. For example, a composition may include barley extract and lemon extract alone. Compositions comprising barley extract and lemon extract have been shown to be effective in the control of fungi. Alternatively, the composition may include barley extract, lemon extract and chelated ions and so on. Wheat extract may also be used as a substitute for the barley extract.

Choline citrate is a safe chelator that functions as a non-metal molecule that is capable of binding the mineral ions thus enhancing their effectiveness. The combination with barley and/or lemon extract provides a safe, “green”, antimicrobial agent.

Without limiting the invention to any particular theory or mechanism of action, it is believed that the barley extract contains polymers that are effective in rendering the cell wall of the microorganism permeable to the passage of the chelated mineral ions. Once the ions are inside the cell they can exert their effects. Special glucans, which are different from those of bacteria, are found in barley and may exert an antimicrobial effect. The lemon or grape extract form the juice or skin may provide essential oils that are antimicrobial. The extracts may form ligands which co-ordinate chemically with the divalent cations. Thus, the combination of components having different effects on the microorganism provides a surprising synergistic effect.

The antimicrobial compositions of the present invention can be provided in a variety of formulations depending on the intended use. For example, the compositions can be formulated as liquids, gels, aerosols, gases, powders, creams or solids (e.g. soap bar, food additive). The antimicrobial compositions may also be formulated to include additional ingredients such as fragrances, thickeners, lubricants, dyes, detergents and other additives apparent to those skilled in the art. The antimicrobial compositions of the present invention may also be provided in combination with other antimicrobial agents (e.g. alcohol, phenol, antibiotics, ammonium, etc.). The compositions of the present invention are effective at a broad range of pH concentrations, from about 2 to about 8.

The compositions of the present invention may be provided in a ready-to-use liquid format or in a concentrated format. The concentrated format is preferably soluble in water. A variety of dilution ratios can be used to generate an effective amount depending on the proposed use so long as the diluted composition exhibits the appropriate antimicrobial activity. The diluted or ready-to-use composition preferably has a concentration of about 2 to about 20%. An 8% solution has been shown to effective in the elimination of 100% of organisms, a 5% solution is effective for about 80% microbial control and a 2% solution provides about 50% reduction in microbial contamination. These numbers refer to reduction of established microorganisms. A 2% solution of the composition of the present invention has been found to be effective in the prevention of microbial contamination.

The ratio of chelated mineral ions to extract of barley may be varied according to the microbial load and/or the substrate to be treated. The chelated mineral ions and barley extract are preferably present in amounts that provide a greater than additive antimicrobial action, both in vivo and in vitro. The ratio of mineral ions to barley extract may be varied. A ratio of 2:1 and 3:1 were found to be effective against bacteria. A ratio of 2.1 is preferably used to prepare a stock solution. Different concentrations of the stock solution have been demonstrated to be effective. In particular, 5%, 10% and 12% were found to be very effective in in vitro studies. Most preferably 10% and 12% are useful in the elimination of higher concentrations of bacteria in the range of greater than 10⁵ cells/ml. The effectiveness of the compositions was tested using various microbiological assays done in liquid nutrient broth cultures, and in surface treatment of meat such as beef and chicken. Different ratios may be effective for other uses. For example, a ratio of 1:3 of ions to barley/lemon has been found to be effective against fungi and yeast.

The composition of the present invention may be provided in the form of a kit. The kit includes chelated metal ions and barley extract. The chelated metal ions and barley extract may be provided together as a concentrate or they may be provided separately to be combined by the end user. The kit will include directions for the preparation of the ready-to-use composition and directions for following the method of the present invention.

The antimicrobial compositions of the present invention are useful in a variety of domestic, industrial, health-related and environmental applications. Due to their low toxicity, as compared to other antimicrobial agents, the compositions of the present invention are particularly useful in the food industry. The compositions can be used on food to reduce or eliminate surface contamination with particular microbes. The compositions are particularly useful for the surface treatment of meats, poultry and fish. The compositions of the present invention are also useful as a coating to protect fruits and vegetables, as well as to prevent diseases (e.g. root rot, leaf spots, etc.) in plants. They are also useful for the reduction of certain bacteria in eggs and to reduce the population of certain microbes in manure. The compositions of the present invention are further useful in the food industry to sanitize food processing equipment such as knives, slicers, tables, sinks, counters and other processing equipment such as pumps, hoses, tanks, vats and the like. The compositions are also useful for coating food-packaging materials such as plastic, metal or glass containers, cartons, wraps, bags, etc.

The compositions of the present invention are also useful in human and animal health related applications. For example, the compositions can be used to treat skin infections and to reduce the spread of skin diseases. They are also useful in the prevention of intestinal disorders caused by mircoorganism infection. The compositions have been shown to be effective against gram-negative organisms such as those that are the causative agents of diseases such as diarrhoea, cholera, meningitis, dysentery, typhoid and food poisoning. They are also effective against gram-positive organism including sporulating organisms and their spores. The compositions can also be used in toothpaste and mouthwash to combat oral microorganisms responsible for caries and halitosis. The compositions can also be used in feedstuffs to reduce the bacteria in manure and the incidence of certain microorganisms in eggs.

Antimicrobial compositions according to the present invention are also useful in a variety of other domestic and industrial applications. Around the house, the compositions can be used to sanitize food preparation surfaces, sinks, bathtubs, toilets, mops, children's toys and the like. A concentrated form of the composition is useful in the decontamination of water and other liquids.

The present invention also provides a method for the decontamination of solid surfaces. The method comprises applying to the solid surfaces an effective low concentration choline citrate chelated metal ions in combination with an extract of barley. In some embodiments, the composition also includes and extract of lemon. The composition may be applied as a s pray, liquid, gel, etc. The composition may be incorporated in a “wet wipe” which used to wipe down a surface.

As used herein, the term “decontaminate” is used to refer to both biostatic and biocidal activities. The words, “sanitize” and “disinfect” are also used interchangeably. The composition eliminates, controls, reduces, and/or prevents the presence of pathogenic bacteria, yeast and filamentous fungi and is also effective against certain viruses. The composition is also effective against spores and sporulating cells. The term “effective amount” is used herein to refer to a concentration sufficient to induce a bactericidal, bacteriostatic, fungicidal or fungistatic effect on a microorganism that is contacted with the composition. The quantity of the composition that determines an effective amount may vary according to the material to be treated and the target microorganism. The terms “contact”, “contacting” and “contacted” are used to refer to situations where the compositions of the present invention come into juxtaposition with the material to be treated.

The present invention provides a method for the treatment of solid and liquid substrates to remove harmful microorgansims. Foods such as meat, fish, vegetables, etc., as well as beverages and water can be safely treated with the compositions of the present invention. The method has been demonstrated to be effective against Salmonella spp, E. coli, Listeria, Staphylococcus, and other pathogenic bacteria. The compositions of the present invention are effective against sporulating bacteria including Bacillus anthracis. Ingestion of food contaminated with Anthrax spores results in acute inflammation of the intestinal tract and 25% to 60% of cases of intestinal anthrax result in death. The antimicrobial composition of the present invention is effective in eliminating both spores and sporulating cells. After a 24-hour treatment, virtually all the spores and bacteria are eliminated.

The compositions of the present invention are also active against yeast such as Candida albicans, Paracoccodiodes, Nocardia, etc. and other pathogenic yeasts. The composition is also useful in the control of filamentous fungi such as Aspergillus, Penicillium, Fusarium, Trychophyton, and other pathogenic moulds. A composition comprising barley extract and lemon extract is particularly effective in the control of fungi.

The present invention addresses the need for alternative ways to enhance the safety of our food. The invention provides a safe alternative to other ways of disinfecting food, such as irradiation, in an environmentally friendly composition that is versatile for a variety of uses.

The above disclosure generally describes the present invention. A more complete understanding can be obtained by reference to the following specific examples. These examples are described solely for purposes of illustration and are not intended to limit the scope of the invention. Changes in form and substitution of equivalence are contemplated as circumstances may suggest or render expedient. Although specific terms have been employed herein, such terms are intended in a descriptive sense and not for purposes of limitation.

### EXAMPLES

The following examples are described for the purposes of illustration only. Methods of microbiology referred to, but not explicitly described in this disclosure and examples are reported in the scientific literature and are well known to those skilled in the art. In certain of the examples, tables and figures the antimicrobial composition of the invention is referred to as Bio-Ion.

### Example 1

### Effect of Antimicrobial Composition on Bacterial Growth in Liquid Culture

Bacterial cultures of Salmonella, E. coli and/or Listeria were mixed with chelated mineral ions alone or in combination with the barley extract for different periods of time and then subjected to microbial analysis. The bacteria analysis was done using standard methods. Spectrophotometry was used for optical density (absorbance) and a dilution plating method was used to count the colony forming units. Agar culture media were used according to the type of bacteria. For example, MacConkey Agar at pH 7.2 was used for E. coli. Green bile Agar at pH 7.0 was used for Salmonella and Palcam Agar media at pH 7.4 was used for Listeria. The cultures were incubated at 30° C. Bacterial sampling was done at various times post treatment. The results are shown in Tables 1 to 3. These results clearly illustrate that the compositions tested had potent antimicrobial effects. An enhanced antimicrobial effect is seen when the barley extract is included with the chelated mineral ions.

TABLE 1
Effect of Cholin Citrate Chelated Mineral Ions with Barley
Extract on Control of Salmonella spp, and E. coli in mixed
Liquid Culture, After 48 Hour and 7 days at 30° C. Incubation
Bacterial Population: Mean ± SD

| | | |
|---|---|---|
| Treatments | O.D. | CFU |

After 48 hours Incubation

| | | |
|---|---|---|
| Salmonella spp + E. coli | 0.64 ± 0.00 | 1.7 × 10⁸ ± 2.6 × 10⁷ |
| (control) | | |
| Salmonella + E. coli + | 0.070 ± 0.00 | 0.00 ± 0.00 |
| CCC Mineral Ions + Barley | | |
| Extract | | |
| Salmonella spp + E. coli + | 0.12 ± 0.00 | 0.00 |
| CCC Mineral Ions | | |

AFTER 7 DAYS

| | | |
|---|---|---|
| Salmonella spp + E. coli | 1.50 ± 0.038 | 5.3 × 10⁷ ± 2.0 C × 10⁷ |
| (control) | | |
| Salmonella spp + CCC | 0.070 ± 0.001 | 0.00 ± 0.00 |
| Mineral Ions + Barley | | |
| Extract | | |
| Salmonella spp + CCC | 0.128 ± 0.004 | 0.00 |
| Mineral Ions | | |

O.D. = Optical density;
CFU = Colony forming units;
CCC = choline citrate chelated
Initial bacterial inoculum (Salmonella spp + E. coli) − 0.50 O.D.
Ratio of CCC mineral ions:Barley extract = 1:1

TABLE 2
Effect of CCC Mineral Ions with Barley Extract on E. coli,
Salmonella spp, and Listeria spp, After 24 hour, 48 h, 72 h
Bacterial Population (O.D., and CFU) Mean ± SD

| | |
|---|---|
| O.D. | CFU |

After 24 hours

| | | |
|---|---|---|
| E. coli Alone (control) | 0.580 ± 0.06 | 2.9 × 10⁸ ± 8.6 × 10⁷ |
| E. coli + CCC mineral | 0.09 ± 0.00 | 0.00 ± 0.00 |
| ions & barley extract (1:1 | | |
| ratio) | | |

After 48 Hours

| | | |
|---|---|---|
| Salmonella spp Alone | 0.619 ± 0.022 | 3.33 × 10⁷ ± 1.52 × 10⁷ |
| (control) | | |
| Salmonella spp + CCC | 0.051 ± 0.001 | 0.00 ± 0.00 |
| mineral ions & barley | | |
| extract *1:1 ratio) | | |

After 72 Hours

| | | |
|---|---|---|
| Salmonella spp Alone | 0.956 ± 0.047 | 5.33 × 10⁸ ± 1.52 × 10⁸ |
| (control) | | |
| Salmonella spp + CCC | 0.056 ± 0.002 | 0.00 ± 0.00 |
| mineral ions & barley | | |
| (1:1 ratio) | | |
| Listeria spp alone | | |
| (control) | | |
| Listeria spp + CCC | | |
| mineral ions & barley | | |
| extract | | |

O.D. = optical density (initial bacterial inoculum O.D = 0.529.
CFU = colony forming units
CCC mineral ions = choline citrate chelated mineral ions.
Ratio of CCC mineral:barley extract = 3:1

TABLE 3
Effect of CCC Mineral Ions on Staphylococcus
aureus, After 24 hours, 72, hours, and 5 days
Bacterial Population (CFU) Mean ± SD

| | |
|---|---|
| O.D. | CFU |

After 48 Hours

| | | |
|---|---|---|
| Staphylococcus aureus Alone | 0.47 ± 0.03 | 7.23 × 10⁸ ± 2.1 × 10⁷ |
| (Control) | | |
| Staphylococcus aureus + | 0.05 ± 0.00 | 0.00 |
| CCC Mineral Ion & Barley | | |
| Extract | | |

After 72 Hours

| | | |
|---|---|---|
| Staphylococcus aureus Alone | 0.86 ± 0.05 | 7.23 × 10⁸ ± 2.08 × 10⁷ |
| (Control) | | |
| Staphylococcus aureus + | 0.05 ± 0.00 | 0.00 |
| CCC Mineral Ions & Barley | | |
| Extract | | |

After 5 days

| | | |
|---|---|---|
| Staphylococcus aureus | 1.10 ± 0.02 | Plates |
| Alone (Control) | | overcrowded |
| | | (>300 colonies/plate) |
| Staphylococcus aureus + | 0.05 ± 0.00 | 0.00 |
| CCC Mineral Ions + Barley | | |
| Extract | | |

### Example 2

### Treatment of Meat with Antimicrobial Composition

Beef and chicken samples were treated with the chelated mineral ions with or without barley extract. The initial bacterial population on the meats was adjusted with different inoculum sizes. Inoculums of 10³, 10⁵, and 10⁶cells/ml were applied by submersion of the meat into the respective bacterial dilution for different times. Exposure was adjusted for 10, 30 and 60 minutes. It was found that 60 minutes exposure was the best to achieve the final desired bacterial population. After inoculation, the meats were transferred to the chelated mineral salts ion treatment with or without barley extract for different time periods (10, 30, 60 minutes). The meats were then subjected to bacterial analysis. For the analysis, the meats inoculated with bacteria were sampled after their respective treatments. Three different sites of the meat were independently swabbed with sterile cotton within 1 cm² area of a sterile template. Each time, the swab was passed for 30 seconds on each meat site. After swabbing the swabs were transferred into nutrient broth tubes and shaken in a vortex for 30 seconds. Aliquots were then taken from each tube for dilution plates according to standard procedures. Agar media was used according to the type of bacteria as described above. The results indicate that the best treatment time was 60 minutes.

In another independent trial, meats were independently subjected to two sequential treatments with the chelated mineral ions with or without barley extract. After the meats were submerged into the respective bacterial inoculums for 60 minutes, they were transferred into two sequential chelated mineral ion compositions with or without barley extract. There was a four-hour difference between each treatment application.

In another trial, meats were inoculated by immersion in bacterial solutions for 60 minutes and then transferred to two chelated mineral ion treatments with or without barley extract. The first treatment was one hour then a second treatment was applied and the samples were incubated at 30′ overnight. All treatments were effective, but the sequential treatment of one hour followed by the second treatment and overnight incubation provided the most effective results.

### Example 3

### Field Trial in Hogs

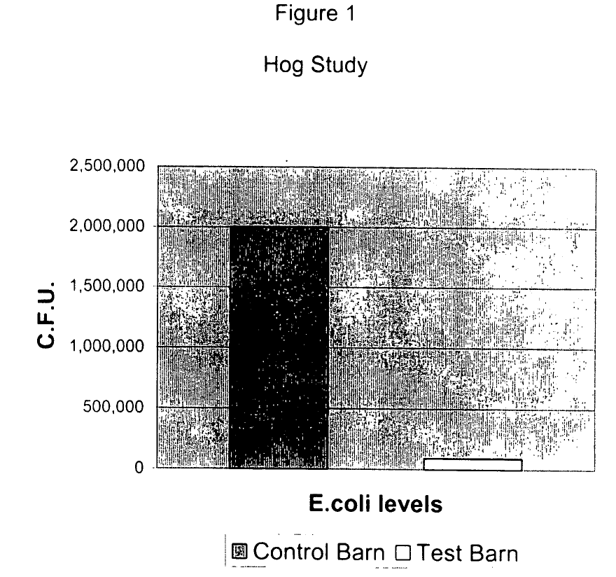

A study was undertaken to determine whether treatment with the antimicrobial composition of the present invention could decrease the population of Salmonella, E. coli, Campylobacter, and Staphylococcus aureus in hog manure. Hogs were treated with the choline citrate chelated mineral ions in mixture with a probiotic containing a lactobacillus fermentation product and barley extract. Eighty hogs were randomly selected and split into forty gills and forty barrows. The hogs were approximately three months old and weighed approximately sixty pounds. The groups were further split into groups of twenty in two pens (20 gills and 20 barrows per pen). Both pens were fed the same with the exception that one pen had the addition of the probiotic which includes barley extract. One kilogram of the dried additive was added per metric ton of feed. Manure was analysed for the presence of Salmonella, E. coli, and Campylobacter using standard microbiological techniques such as plate dilution with specific agar media according to the type of microorganism. The microbial population in the samples was determined using the colony forming units per gram of sample. For Salmonella, the measurement unit was based on the number of organisms per 25 grams. The samples were analysed quantitatively for E. coli, Staphylococcus aureus, yeast and mould. Qualitative analysis only was done for Salmonella and Campylobacter. The results shown in FIG. 1 demonstrate that the hogs in the test barn had significantly lower levels of E. coli in their manure as compared to the control animals.

### Example 4

### Control of Bacterial Spores

Bacillus subtilis was grown in nutrient broth and on nutrient agar plates at pH 6.0. The media were inoculated with a bacterial dilution equivalent to an Optical Density of 0.140 after a spectrophotometric reading. The cultures were incubated at 37° C. under standard shake conditions for liquid cultures. Bacterial growth was determined by optical density, and microscopic observation during one week. Total bacterial growth (vegetative cells+spores), and production of spores, was determined. Spores were counted under a compound microscope at 10× by observing 12 fields.

Total growth was determined by measuring the OD at 520 nm. for 3 replicates in each group. After 24 hours, the control samples had an average OD of 0.22. The treated samples (10% Bio-Ion+Barley extract+Lemon extract) had an average OD of 0.04. After one week, the control samples had an average OD of 0.61 and the treated samples had an average OD of 0.04. These results indicate that the treatment was very effective in controlling bacterial growth.

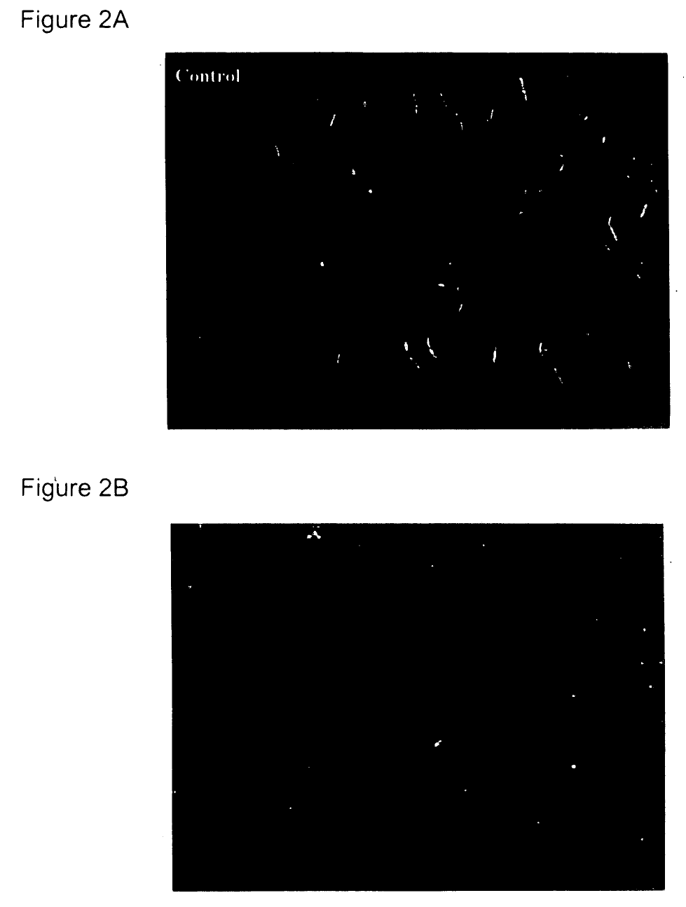

The average spore count for each replicate was determined by twelve microscopic field observations at 10× magnification. Three replicates in each group were counted and the average determined. After 12 hours, the average spore count in the control group was 3.67 and the average spore count in the treated group was 0.17. After one week, the average spore count in the control group was 2.67 and the average spore count in the treated group was 0.50. These results indicate that the formulations of the present invention are also useful in the control of spores from sporulating bacteria. FIG. 2A is a photograph illustrating the control culture and FIG. 2B is a photograph illustrating the treated group.

### Example 5

### Illustration of Control of Vegetative Cells and Spores

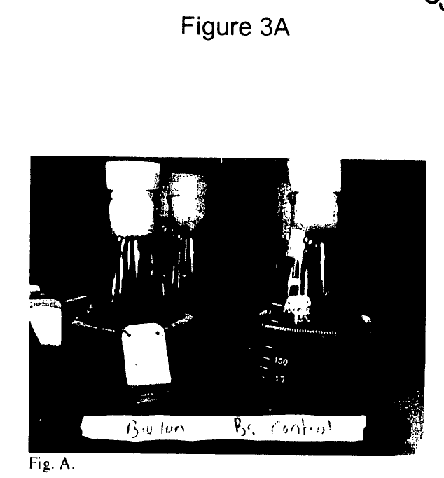

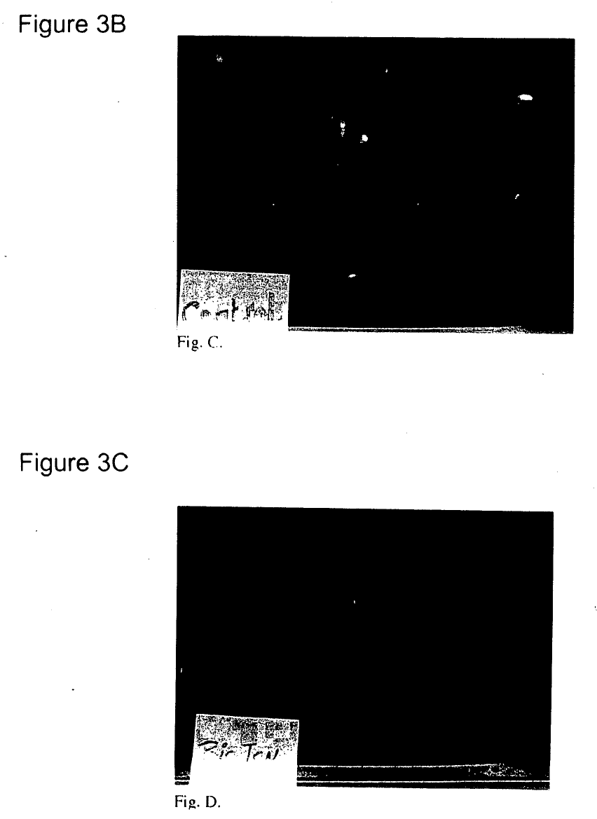

Cultures were prepared as described above. The Bacillus subtilis cultures were divided into control and treatment groups. After 24 hours, the cultures were observed under a microscope and photographed. FIG. 3A shows the effect in liquid culture of the composition of the present invention. The treated culture is shown on the left and the control culture is shown on the right. FIG. 3B is a high power dark field micrograph of a control culture. Both spores and vegetative bacteria can be seen. FIG. 3C is a high power dark field micrograph of a treated culture. There are no vegetative cells or spores remaining in the treated sample. The few spots showing are remnants of lysed cells. These results confirm the efficacy of the formulations of the present invention.

### Example 6

### Control of Sporulating Bacteria

The effect of treatment with an anti-microbial composition according to the present invention on the growth of Bacillus subtilis bacteria and spores was assessed over several days. The results are shown below:

| | COLONY FORMING UNIT (CFU) | | |
|---|---|---|---|
| | Day 1 | Day 5 | Day 6 |
| TREATMENT | Mean ± SD | Mean ± SD | Mean ± SD |
| B. subtilis | 4 × 10⁶ ± 1.00 | 7.7 × 10⁷ ± 1.53 | 1 × 10⁸ ± 0.00 |
| B. subtilis + Bio- | 0.00 | 0.00 | 0.00 |
| Ion | | | |

### Example 7

### Effective Control of E. coli in Chicken

Whole chicken was soaked in warm water for 1 hour. Then the water was let to drip for 30 minutes. Chicken was then inoculated by soaking the whole chicken for 1 hour with solution of bacteria E. coli with a population equivalent to 0.110 optical density (O.D.) as determined in the spectrometer at wave-length set at 539 nm. Chicken was let to drip for 1.5 hour. The initial bacterial population (before inoculation) of the chicken was 3×10² cells/ml CFU. In the treatment group, whole chicken was immersed in Bio-Ion (3:1 Chelated Ions:Extract of barley and lemon) overnight. Control chicken was not immersed in Bio-Ion. The following day, chicken skin or surface was sampled using the swabbing technique. The bacterial population was analyzed by CFU and the results are shown below:

| Treatment | CFU | Mean ± SD |
|---|---|---|
| E. coli Alone (Control) | 5 × 10⁷ | =3.33 × 10⁷ ± 1.88 × 10⁷ |
| | 4 × 10⁷ | |
| | 7 × 10⁷ | |
| | 8 × 10⁷ | |
| | 3 × 10⁷ | |
| | 5 × 10⁷ | |
| E. coli + Bio-Ion | 7 × 10² | =5.5 × 10² ± 2 × 10² |
| | 6 × 10² | |
| | 8 × 10² | |
| | 2 × 10² | |
| | 5 × 10² | |
| | 5 × 10² | |

1. An anti-microbial composition comprising a barley extract and at least one chelated mineral salt.

2. The composition of claim 1 wherein the barley extract is derived from organically grown barley.

3. The composition of claim 1 wherein the chelated mineral salt includes a metal ion selected from the group consisting of iron, zinc, copper, cobalt, magnesium, manganese and mixtures thereof.

4. The composition of claim 2 wherein the metal ion is iron.

5. The composition of claim 1 comprising a ratio of chelated mineral salt to barley extract selected from the group consisting of 1:3, 1:2, 1:1.5 and 3:1.

6. The composition of claim 1 further comprising lemon extract or grape extract.

7. The composition of claim 5 comprising a chelated mineral salt to barley extract to lemon ratio of 1:1.5:0.5.

8. The composition of claim 1 formulated as a liquid, spray, gel, cream or solid.

9. A method of reducing contamination with bacteria, bacterial spores, yeast and fungi comprising applying a composition as defined in claim 1.

10. The method of claim 9, wherein the composition is applied to a product selected from the group consisting of meat, fish, poultry, fruits, vegetables, beverages and water.

11. The method of claim 9 wherein the composition is applied to a food-handling surface.

12. The method of claim 11 wherein the food handling surface is selected from the group consisting of food preparation equipment, table, sink, counter, food wrap and food containers.

13. The method of claim 9, wherein the composition is applied to a body surface.

14. The method of claim 13, wherein the body surface is selected from the group consisting of skin, teeth and mucosal surfaces.

15. A method of reducing microbial load in an animal comprising feeding to said animal a composition as defined in claim 1.
